(19) United States
(12) Patent Application Publication
(10) Pub. No.: US 2024/0318967 A1
(43) Pub. Date: Sep. 26, 2024

(54) RECOMMENDED ACTIVITY PRESENTATION DEVICE, RECOMMENDED ACTIVITY PRESENTATION SYSTEM, RECOMMENDED ACTIVITY PRESENTATION METHOD, AND STORAGE MEDIUM STORING RECOMMENDED ACTIVITY PRESENTATION PROGRAM

(71) Applicant: Mitsubishi Electric Corporation, Tokyo (JP)

(72) Inventors: Nobuyuki KOIZUMI, Tokyo (JP); Takuya MAEKAWA, Tokyo (JP); Kaori HANDA, Tokyo (JP)

(73) Assignee: Mitsubishi Electric Corporation, Tokyo (JP)

(21) Appl. No.: 18/732,760

(22) Filed: Jun. 4, 2024

Related U.S. Application Data
(63) Continuation of application No. PCT/JP2021/048170, filed on Dec. 24, 2021.

(51) Int. Cl. G01C 21/34; A61B 5/0205; A63B 24/00; G01C 21/36

(52) U.S. Cl. CPC G01C 21/3461; A61B 5/02055; A63B 24/0075; G01C 21/3602; G01C 21/3626

(57) ABSTRACT

A recommended activity presentation device for managing an activity amount of an object person, includes processing circuitry to determine a recommended course as a movement course recommended to the object person among the plurality of movement courses that exist in a structure and can be used by the object person, or a recommended speed as a walking speed recommended when moving through the movement course, or both of the recommended course and the recommended speed based on the shortage activity amount, and to output the notification information as the recommended course or the recommended speed or both of the recommended course and the recommended speed, to a display device for presenting notification information to the object person.

### CROSS-REFERENCE TO RELATED APPLICATION

This application is a continuation application of International Application No. PCT/JP2021/048170 having an international filing date of Dec. 24, 2021.

### BACKGROUND OF THE INVENTION

### 1. Field of the Invention

The present disclosure relates to a recommended activity presentation device, a recommended activity presentation system, a recommended activity presentation method, and a recommended activity presentation program.

### 2. Description of the Related Art

There has been proposed an information output system including an acquisition unit to acquire physical condition information regarding physical condition of a user before starting activity (e.g., workout) and an output unit that presents a recommended route, recommended to the user as a route through which the activity should be done (e.g., a route of running or walking), based on the physical condition information (see Patent Reference 1, for example). This system is targeted for users doing a workout at a workout facility.

Patent Reference 1: Japanese Patent Application Publication No. 2018-023681 (see claim 1 and abstract, for example).

However, in recent years, in structures such as office buildings (e.g., wellness buildings), there is a demand for a device capable of individually presenting recommended activity to people in order to mitigate insufficiency of physical activity amounts of people using the structure. Here, the wellness is a concept aiming to healthily lead the everyday life while properly incorporating exercise into the daily life.

### SUMMARY OF THE INVENTION

An object of the present disclosure is to provide a recommended activity presentation device, a recommended activity presentation system, a recommended activity presentation method and a recommended activity presentation program that make it possible to present notification information for mitigating the insufficiency of the activity amount of an object person using a structure to the object person.

A recommended activity presentation device for managing an activity amount of an object person, including processing circuitry to determine a recommended course as a movement course recommended to the object person among the plurality of movement courses that exist in a structure and can be used by the object person, or a recommended speed as a walking speed recommended when moving through the movement course, or both of the recommended course and the recommended speed based on the shortage activity amount, and to output the notification information as the recommended course or the recommended speed or both of the recommended course and the recommended speed, to a display device for presenting notification information to the object person.

A recommended activity presentation method in the present disclosure is a method to be executed by a recommended activity presentation device for managing an activity amount of an object person. The method includes determining a recommended course as a movement course recommended to the object person among the plurality of movement courses that exist in a structure and can be used by the object person, or a recommended speed as a walking speed recommended when moving through the movement course, or both of the recommended course and the recommended speed based on the shortage activity amount, and outputting the notification information as the recommended course or the recommended speed or both of the recommended course and the recommended speed, to a display device for presenting notification information to the object person.

According to the present disclosure, the notification information for mitigating the insufficiency of the activity amount of the object person using the structure can be presented to the object person.

### DETAILED DESCRIPTION OF THE INVENTION

Further scope of applicability of the present invention will become apparent from the detailed description given hereinafter. However, it should be understood that the detailed description and specific examples, while indicating preferred embodiments of the invention, are given by way of illustration only, since various changes and modifications will become apparent to those skilled in the art from the detailed description,

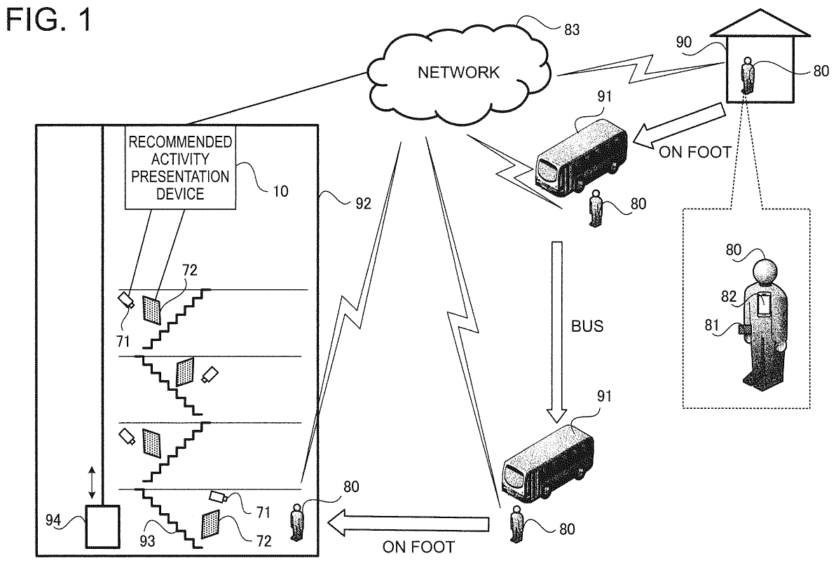

FIG. 1 is a schematic diagram showing an example of application of a recommended activity presentation device according to a first embodiment;

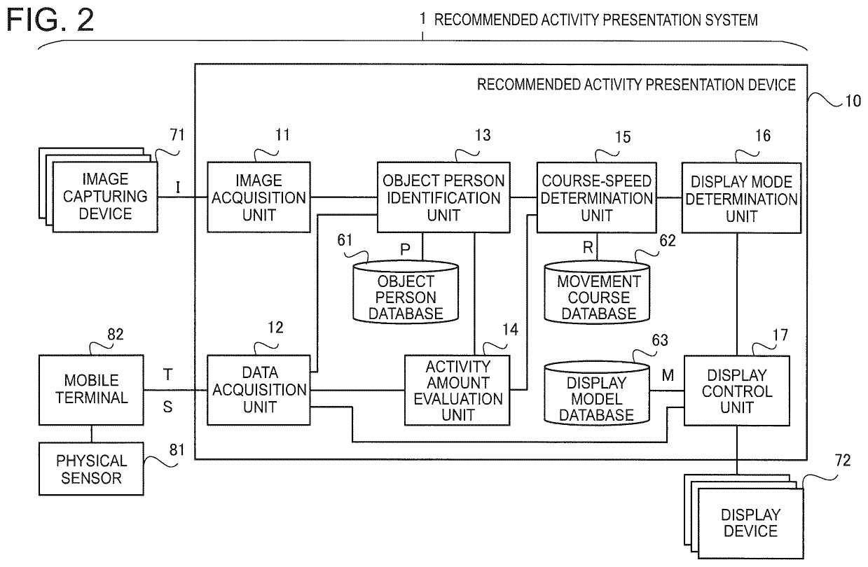

FIG. 2 is a functional block diagram schematically showing the configuration of the recommended activity presentation device and a recommended activity presentation system according to the first embodiment;

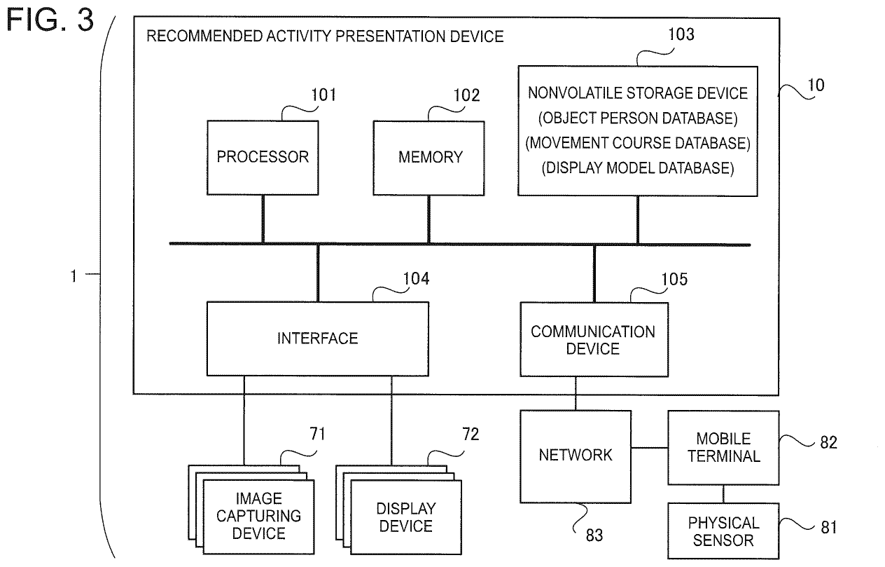

FIG. 3 is a diagram showing an example of the hardware configuration of the recommended activity presentation device and the recommended activity presentation system according to the first embodiment;

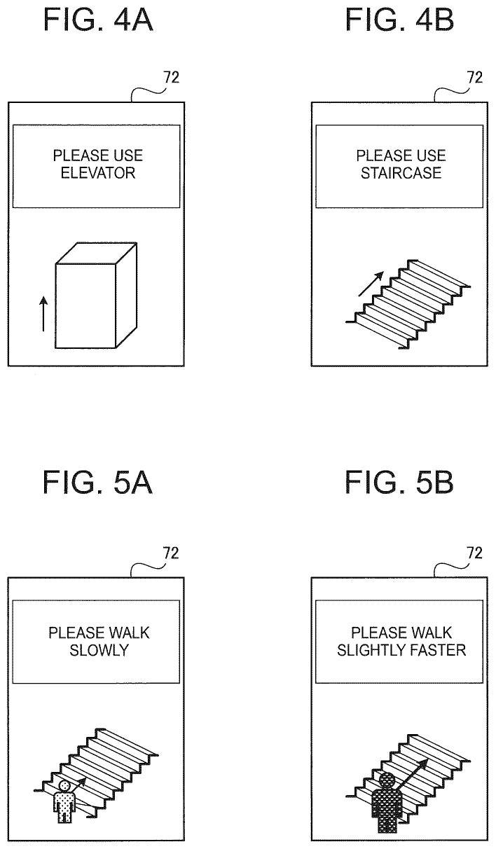

FIGS. 4A and 4B are diagrams showing display examples of a display device;

FIGS. 5A and 5B are diagrams showing other display examples of the display device;

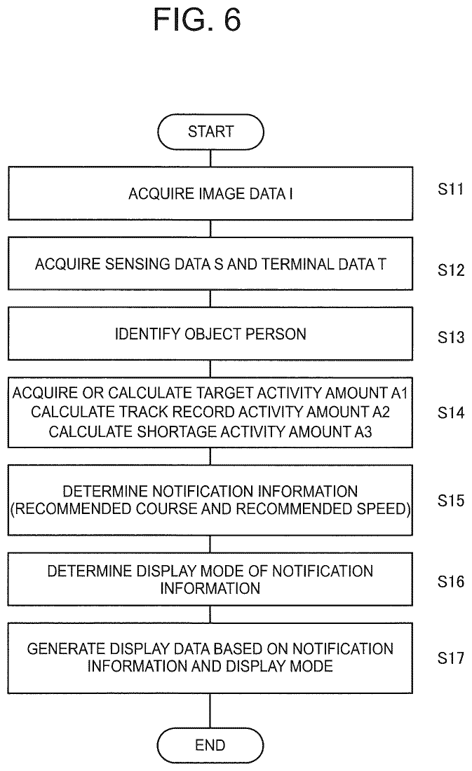

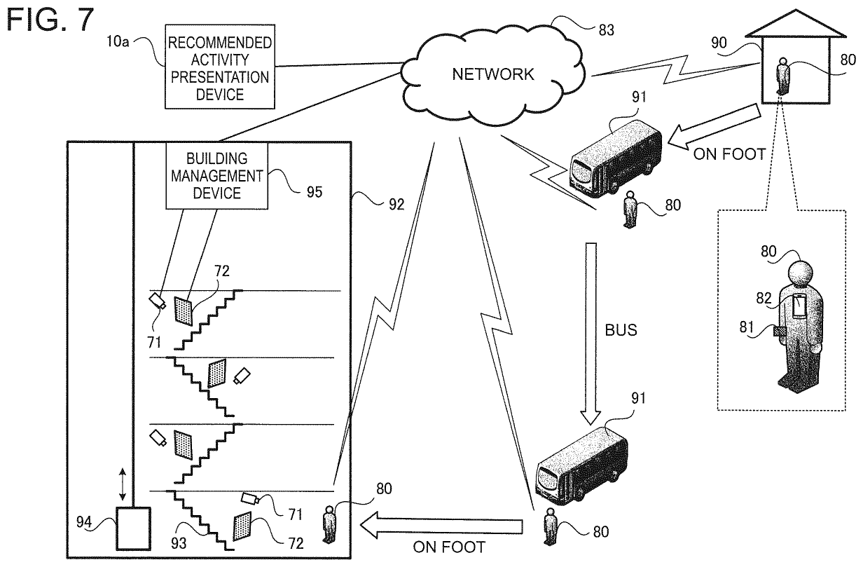

FIG. 6 is a flowchart showing the operation of the recommended activity presentation device according to the first embodiment;

FIG. 7 is a schematic diagram showing an example of application of a recommended activity presentation device according to a first modification of the first embodiment;

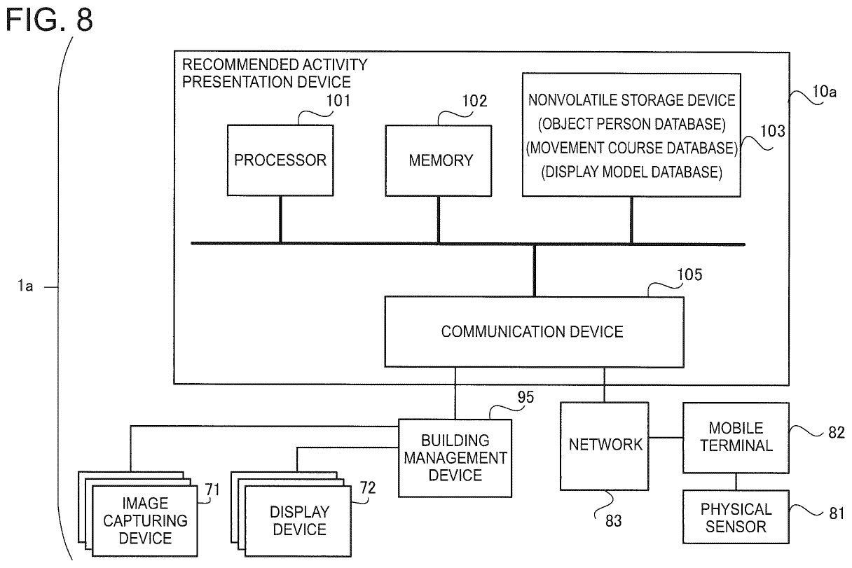

FIG. 8 is a functional block diagram schematically showing the configuration of the recommended activity presentation device and a recommended activity presentation system according to the first modification of the first embodiment;

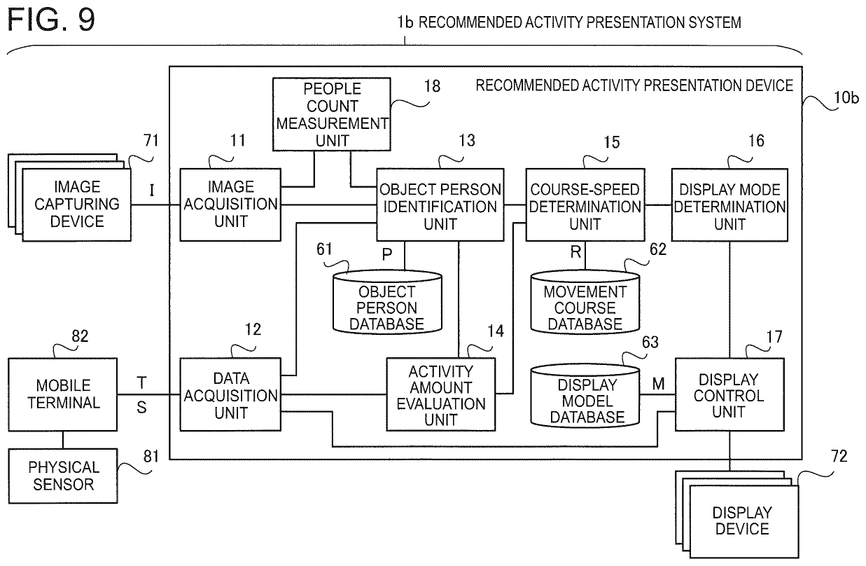

FIG. 9 is a functional block diagram schematically showing the configuration of a recommended activity presentation device and a recommended activity presentation system according to a second modification of the first embodiment;

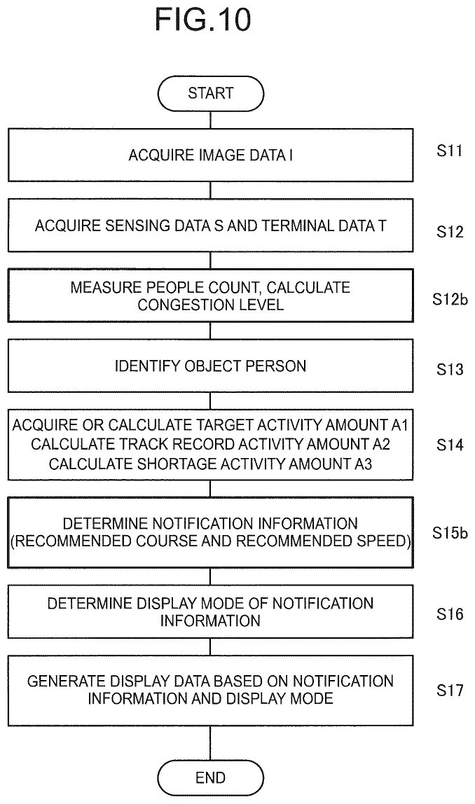

FIG. 10 is a flowchart showing the operation of the recommended activity presentation device according to the second modification of the first embodiment;

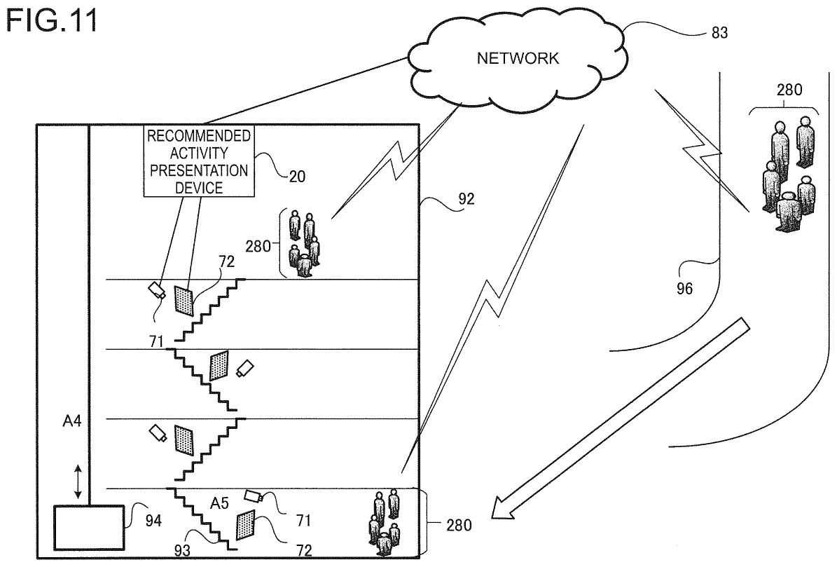

FIG. 11 is a schematic diagram showing an example of application of a recommended activity presentation device according to a second embodiment;

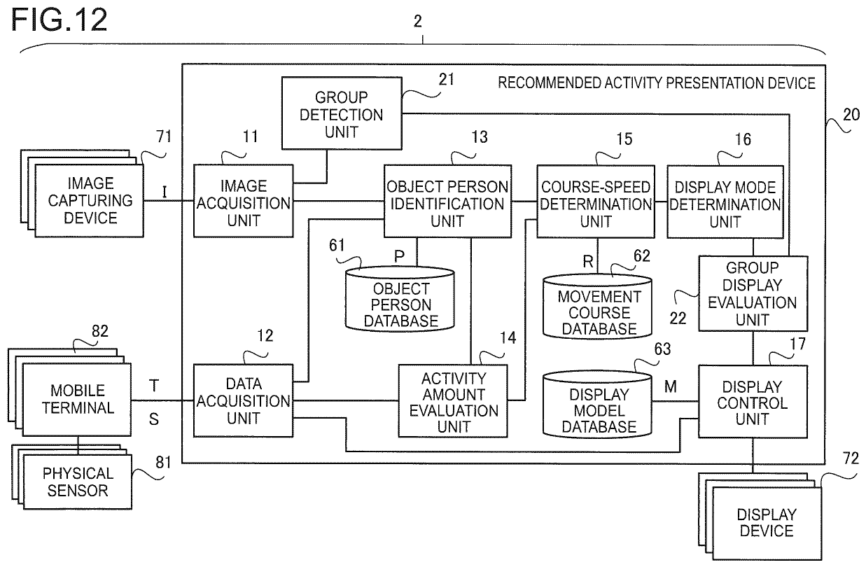

FIG. 12 is a functional block diagram schematically showing the configuration of the recommended activity presentation device and a recommended activity presentation system according to the second embodiment;

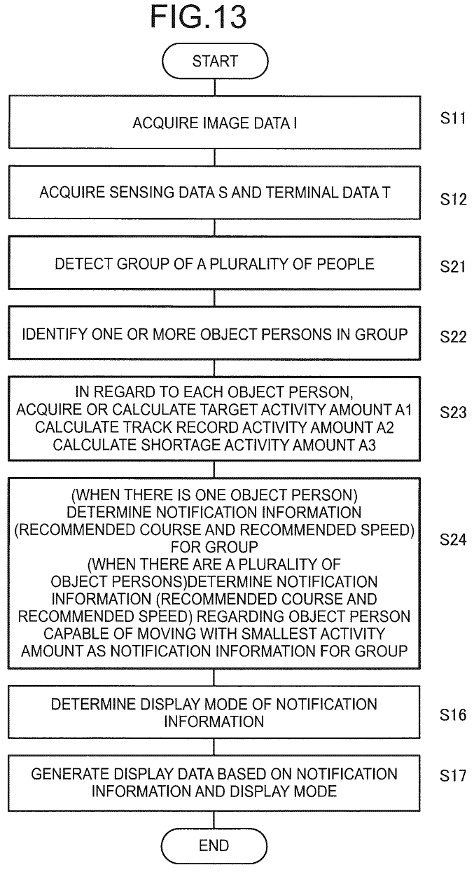

FIG. 13 is a flowchart showing the operation of the recommended activity presentation device according to the second embodiment;

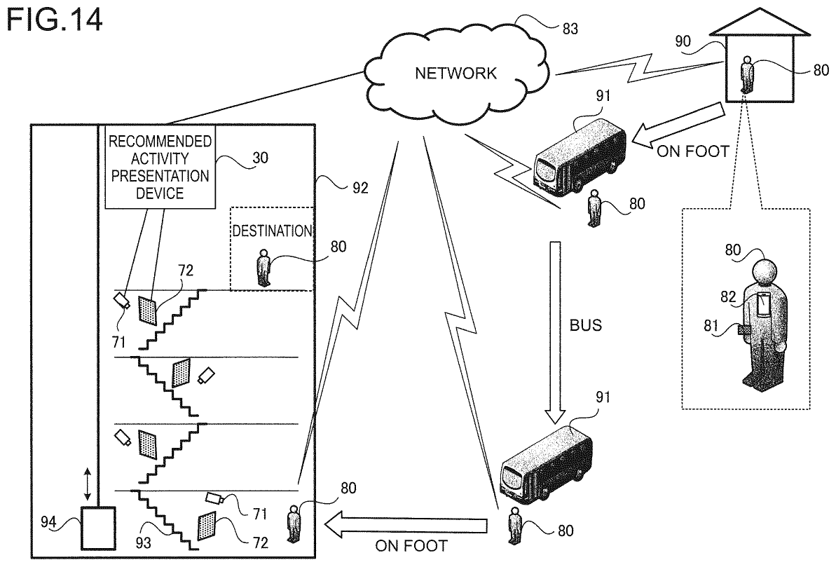

FIG. 14 is a schematic diagram showing an example of application of a recommended activity presentation device according to a third embodiment;

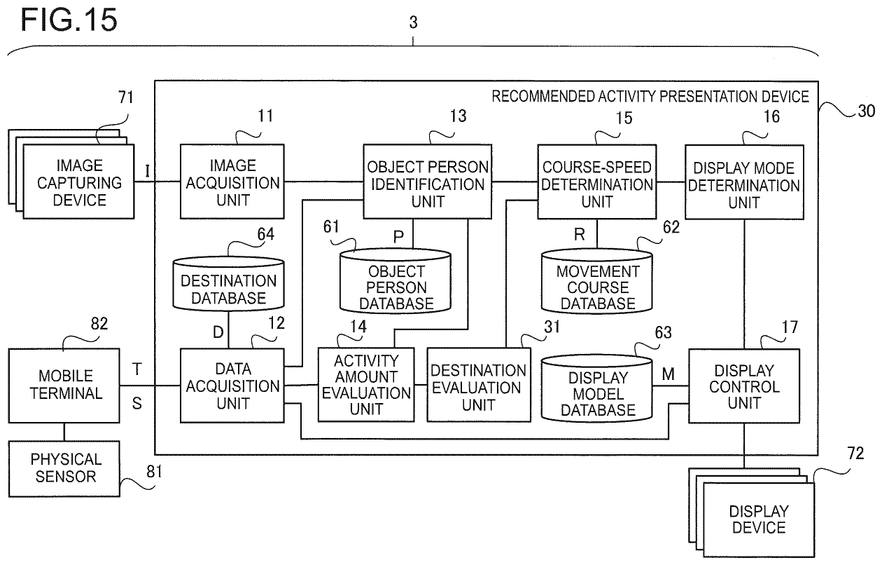

FIG. 15 is a functional block diagram schematically showing the configuration of the recommended activity presentation device and a recommended activity presentation system according to the third embodiment; and

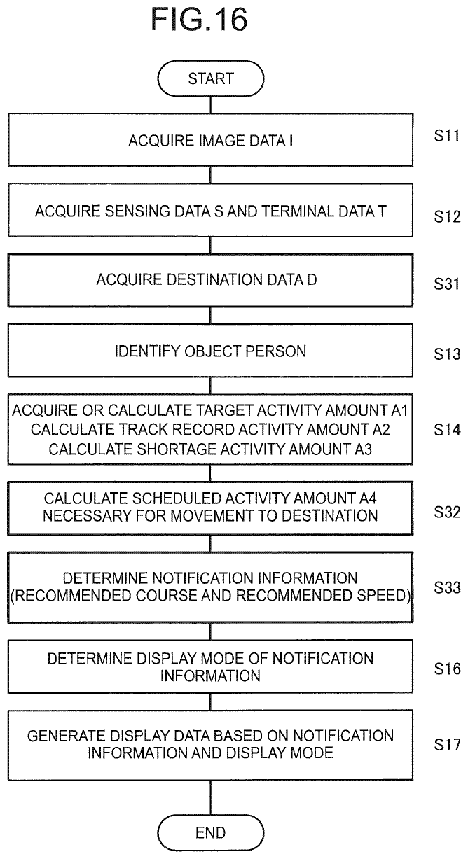

FIG. 16 is a flowchart showing the operation of the recommended activity presentation device according to the third embodiment.

### DETAILED DESCRIPTION OF THE INVENTION

A recommended activity presentation device, a recommended activity presentation system, a recommended activity presentation method and a recommended activity presentation program according to each embodiment will be described below with reference to the drawings. The following embodiments are just examples and it is possible to appropriately combine embodiments and appropriately modify each embodiment.

### First Embodiment

FIG. 1 is a schematic diagram showing an example of application of a recommended activity presentation device 10 as an activity amount management device according to a first embodiment. FIG. 1 shows a state in which an object person 80 as a person whose physical activity amount (hereinafter referred to also as an “activity amount”) is managed by the recommended activity presentation device 10 moved on foot from home 90 to a bus stop, rode on a bus 91, thereafter got off the bus 91, and arrived on foot at an entrance of a structure (e.g., a wellness building as an office building) 92 accommodating an office as the workplace after getting off the bus 91.

The object person 80 carries a physical sensor 81 and a mobile terminal 82. The physical sensor 81 is a wearable sensor that is attached to the object person 80 and senses physical condition of the object person 80. The physical sensor 81 is, for example, a device of the wristwatch type attached to a wrist. However, the physical sensor 81 can also be a device of a different type, such as a device hanging from the neck or a device embedded in clothing. The physical sensor 81 senses, for example, one of more (i.e., one, or a set of two or more) out of the number of steps of the object person 80, body temperature of the object person 80 and a heart rate of the object person 80. The physical quantity regarding the body sensed by the physical sensor 81 can also be a physical quantity other than the number of steps, the body temperature or the heart rate as long as the physical quantity makes it possible to calculate the activity amount (corresponding to energy consumption) of the object person 80.

The mobile terminal 82 is, for example, a smartphone, a mobile phone, a tablet terminal, a small-sized personal computer, or the like. The mobile terminal 82 is a device capable of communicating with the recommended activity presentation device 10 via a network 83 such as the Internet. The physical sensor 81 executes communication with the recommended activity presentation device 10 via the mobile terminal 82 having the communication function. That is, the physical sensor 81 transmits sensing data S to the mobile terminal 82, and the mobile terminal 82 transmits the sensing data S to the recommended activity presentation device 10. However, the physical sensor 81 can also be a device capable of directly executing the communication via the network 83. Further, the physical sensor 81 can also be a part of the mobile terminal 82. In FIG. 1, the physical condition is transmitted to the recommended activity presentation device 10 not only in periods when the object person 80 is inside the structure 92 but also in a period when the object person 80 moves from the home 90 to the structure 92. Further, it is also permissible even if the physical condition is transmitted after the movement of the object person. Therefore, the recommended activity presentation device 10 is capable of calculating a track record activity amount A2 as the activity amount consumed until the time point of arriving at the structure 92.

The recommended activity presentation device 10 is installed in the structure 92, for example. The structure 92 includes one or a plurality of movement courses (i.e., guidance courses) that can be used by the object person 80. The movement courses include a staircase 93, an elevator 94 and an escalator used by people when moving upstairs or downstairs and one or a plurality of corridors interconnecting areas on the same floor separate from each other. Further, one or more image capturing devices and one or more display devices 72 are arranged in the structure 92. The one or more image capturing devices may include a first image capturing device arranged in the vicinity of the entrance of the structure 92, a second image capturing device arranged in the vicinity of a boarding point of the elevator 94, and a third image capturing device arranged in the vicinity of a landing of the staircase 93. The one or more display devices 72 are devices like digital signage arranged at the same places as the one or more image capturing devices 71. However, the display devices 72 and the image capturing devices 71 do not necessarily have to be arranged at the same places but can be arranged at different places. The display device 72 presents notification information to the object person 80. The image capturing device 71 is a camera for capturing video. The image capturing device 71 can also be a camera for capturing still images. The image capturing device 71 captures an image of a person such as the object person 80. The image capturing device 71 outputs image data I. The display device 72 is, in general, a liquid crystal display device. The display device 72 can also be a projector that projects an image onto a floor, a wall, a ceiling or the like of the structure 92.

FIG. 2 is a functional block diagram schematically showing the configuration of the recommended activity presentation device 10 and a recommended activity presentation system 1 as an activity amount management system according to the first embodiment. The recommended activity presentation device 10 is a device capable of executing a recommended activity presentation method as an activity amount management method according to the first embodiment. The recommended activity presentation device 10 is, for example, a computer capable of executing a recommended activity presentation program as an activity amount management program according to the first embodiment.

The recommended activity presentation system 1 is a system for managing the activity amount of the object person 80. In FIG. 2, the object person 80 is a person who has been registered in an object person database 61. The recommended activity presentation system 1 includes one or a plurality of image capturing devices 71 to capture images of the inside of the structure 92 that can be used by the object person 80, one or a plurality of physical sensors 81 sensing the physical condition of one or a plurality of object persons 80, one or a plurality of mobile terminals 82 carried by one or a plurality of object persons 80, one or a plurality of display devices 72 provided in the structure 92, and the recommended activity presentation device 10 managing the activity amount of the object person 80.

The recommended activity presentation device 10 includes an image acquisition unit 11, a data acquisition unit 12, an object person identification unit 13, an activity amount evaluation unit 14, a course-speed determination unit 15, a display mode determination unit 16 and a display control unit 17. The recommended activity presentation device 10 includes the object person database 61, a movement course database 62 and a display model database 63. Part or all of these databases may be stored in a storage device outside the recommended activity presentation device 10.

The image acquisition unit 11 acquires the image data I outputted from the one or more image capturing devices 71 capturing images of the inside of the structure 92. The data acquisition unit 12 acquires the sensing data S outputted from the physical sensors 81 sensing the physical condition of the object persons 80 and terminal data T for identifying the mobile terminals 82 carried by the object persons 80.

The object person identification unit 13 identifies (i.e., determines) each object person 80 based on the image data I obtained by the image capturing of the object person 80 and object person data P stored in the object person database 61 and indicating attributes of the object persons 80. Alternatively, the object person identification unit 13 may also identify (i.e., determine) each object person 80 based on the terminal data T regarding the mobile terminal 82 carried by the object person 80 and the object person data P stored in the object person database 61. The object person data P includes information indicating age, sex, body weight and the like of each object person 80, for example, as the attributes. Further, the object person data P includes, for example, information indicating a handicap, disease (i.e., chronic disease), or a handicap and disease that the object person 80 has. Furthermore, the object person data P may include information indicating a travel distance from a start point (e.g., the home 90) of the object person 80 to the structure 92 (e.g., the workplace) and a transportation system used by the object person 80.

The activity amount evaluation unit 14 acquires or calculates a target activity amount A1 of the object person 80 per predetermined reference period (e.g., per day) based on the object person data P (e.g., the age, the sex, the body weight and the like) stored in the object person database 61. The target activity amount A1 is the amount of energy that the object person 80 is desired to consume per day. Further, the activity amount evaluation unit 14 calculates the track record activity amount A2, as the activity amount of the object person 80 from a start time point of the reference period (e.g., one day) to the present time point, based on the sensing data S and the object person data P. The track record activity amount A2 is, for example, the amount of energy that the object person 80 consumed from the beginning (twelve o'clock midnight) of one day to the present time point. The activity amount evaluation unit 14 calculates a shortage activity amount A3 as an activity amount by which the object person 80 is short based on the target activity amount A1 and the track record activity amount A2. For example, the activity amount evaluation unit 14 calculates the shortage activity amount A3 by subtracting the track record activity amount A2 from the target activity amount A1. The target activity amount A1, the track record activity amount A2 and the shortage activity amount A3 are amounts of energy (unit: [METs·HR]).

For example, the activity amount evaluation unit 14 calculates the shortage activity amount A3 by evaluating how much the track record activity amount A2 of one day is short as compared to the target activity amount A1 based on the sensing data S acquired from the physical sensor 81. The activity amount is calculated by, for example, multiplying “METs” as an index of exercise intensity by an exercise time HR [hr] (i.e., calculated as METs·HR). The MET (metabolic equivalent) is an amount obtained by dividing energy consumption in physical activity by metabolism in a seated resting state (corresponding to approximately 3.5 [ml/kg/min] in terms of the oxygen intake). In other words, the METs is an amount indicating that the intensity of the exercise corresponds to how many times “1 MET” that is assumed to be the METs in the resting state. For example, walking corresponds to 3.0 METs and fast walking corresponds to 4.0 METs.

The course-speed determination unit 15 determines a movement course recommended to the identified object person 80 among the plurality of movement courses in the structure 92 (referred to also as a “recommended course”), a recommended walking speed when moving through the movement course (referred to also as a “recommended speed”), or both of the recommended course and the recommended speed based on the shortage activity amount A3 and the object person data P regarding the identified object person 80. For example, the course-speed determination unit 15 evaluates to which of a movement course through which an exercise amount is large and a movement course through which the exercise amount is relatively small the object person 80 should be guided based on the acquired sensing data. The magnitude of the exercise amount is set by an installer when the recommended activity presentation device is installed. When the display device is placed at a branch point of a passage leading to the staircase 93 and a passage leading to the elevator 94, the passage leading to the staircase is set as a movement course through which the exercise amount is large and the passage leading to the elevator 94 is set as a movement course through which the exercise amount is small. In cases where the object person data P indicates that the object person 80 is a handicapped person or a person having chronic disease, the course-speed determination unit 15 preferentially recommends a movement course including the elevator 94.

The plurality of movement courses include a first course using the staircase 93 of the structure 92 and a second course using the elevator 94 of the structure 92. Further, the plurality of movement courses may include a third course using both of the staircase 93 and the elevator 94 of the structure 92. For example, in the third course, the object person 80 uses the elevator 94 to a floor lower than a floor of the destination and thereafter moves to the floor of the destination by using the staircase 93. Alternatively, in the third course, the object person 80 climbs up the staircase 93 to a floor lower than the floor of the destination and thereafter moves to the floor of the destination by using the elevator 94. The course-speed determination unit 15 may acquire candidates for the movement course from the movement course database 62 previously storing movement course data R and determine the movement course by referring to the movement course data R.

Further, when the shortage activity amount A3 is large (e.g., larger than or equal to a predetermined reference value), the course-speed determination unit 15 recommends the use of the staircase 93 to the object person 80, recommends a fast walking speed to the object person 80, or recommends both of the use of the staircase 93 and the fast walking speed (fast walking) to the object person 80. While the speed of normal walking is 4 km/hr or approximately 4 km/hr, the speed of fast walking is 5 to 6 km/hr or approximately 5 to 6 km/hr, for example, and the speed of slowish walking is 3 km/hr or less, for example. Furthermore, when the shortage activity amount A3 is small (e.g., smaller than a predetermined reference value) , the course-speed determination unit 15 recommends the use of the elevator 94, recommends a slowish walking speed (i.e., speed less than or equal to the speed of the normal walking), or recommends both of the use of the elevator 94 and the slowish walking speed to the object person 80. Moreover, when the object person 80 is judged based on the object person data P in the object person database 61 to be a handicapped person (e.g., wheelchair user) or a person having chronic disease, the course-speed determination unit 15 may determine a movement course using the elevator 94 (i.e., movement course through which movement is possible with an activity amount less than that when using the staircase) as the recommend course irrespective of the value of the shortage activity amount A3.

The display mode determination unit 16 determines a display mode of the notification information to be used when making the display device 72 in the structure 92 display the notification information indicating at least one of the recommended course and the recommended speed. For the determination of the display mode of the notification information, the display mode determination unit 16 may refer to display model data M in the display model database 63. The display mode determination unit 16 determines the mode to be used when displaying the notification information, indicating notification such as through what movement course and at approximately what speed the object person 80 should do the walking, based on the shortage activity amount A3 of the object person 80 guided to a certain movement course. As the display mode, there are a display mode in which the object person 80 climbing up the staircase is guided forward by a leader (i.e., display mode in which the back view of the leader climbing up the staircase is displayed on the screen of the display device 72), a display mode in which the motion of legs of the displayed leader is fast or slow, a display mode in which the leader on the display device 72 guiding the object person 80 is an animal or a character other than a human, and so forth.

The display control unit 17 makes the display device 72 display the notification information, indicating at least one of the recommended course and the recommended speed determined by the course-speed determination unit 15, in the display mode determined by the display mode determination unit 16. The display control unit 17 extracts display model data M that matches the movement course for guiding the guidance object person, the display mode of the image, and the recommended speed, and controls on what display device (s) 72 the notification information should be displayed. In this case, the display model data M is displayed so as to lead and guide the guidance object person at a constant speed in front of the guidance object person. In cases where the leading guidance display is carried out with a plurality of display devices installed in a plurality of areas, it is necessary to prevent the guidance object person from being left behind. Therefore, the display control unit 17 constantly acquires position information regarding the guidance object person.

FIG. 3 is a diagram showing an example of the hardware configuration of the recommended activity presentation device 10 and the recommended activity presentation system 1 according to the first embodiment. As shown in FIG. 3, the recommended activity presentation device 10 includes a processor 101 such as a CPU (Central Processing Unit), a memory 102 as a volatile storage device, a nonvolatile storage device 103 such as a hard disk drive (HDD) or a solid state drive (SSD), an interface 104, and a communication device 105. The memory 102 is a semiconductor memory such as a RAM (Random Access Memory), for example. The object person database 61, the movement course database 62 and the display model database 63 shown in FIG. 2 have been stored in the nonvolatile storage device 103.

Functions of the recommended activity presentation device 10 are implemented by processing circuitry, for example. The processing circuitry can be either dedicated hardware or the processor 101 executing a program stored in the memory 102 as a storage device. The storage device may be a non-transitory computer-readable storage medium, namely, a non-transitory tangible storage medium storing a program such as the recommended activity presentation program. The processor 101 can be any one of a processing device, an arithmetic device, a microprocessor, a microcomputer and a DSP (Digital Signal Processor).

In the case where the processing circuitry is dedicated hardware, the processing circuitry is, for example, a single circuit, a combined circuit, a programmed processor, a parallelly programmed processor, an ASIC (Application Specific Integrated Circuit), an FPGA (Field-Programmable Gate Array) or a combination of some of these circuits.

In the case where the processing circuitry is the processor 101, the recommended activity presentation program to be executed by the recommended activity presentation device 10 is implemented by software, firmware or a combination of software and firmware. The recommended activity presentation program is installed in the recommended activity presentation device 10 via a network or from a record medium. The software and the firmware are described as programs and stored in the memory 102. The processor 101 is capable of implementing the functions of the units shown in FIG. 2 by reading out and executing the recommended activity presentation program stored in the memory 102.

Incidentally, it is also possible to implement part of the recommended activity presentation device 10 by dedicated hardware and part of the recommended activity presentation device 10 by software or firmware. As above, the processing circuitry is capable of implementing the above-described functions by hardware, software, firmware or a combination of some of these means.

FIGS. 4A and 4B are diagrams showing display examples of the display device 72. FIG. 4A shows an example in which the display control unit 17 displays the elevator 94 in order to notify of a movement course using the elevator 94 as the recommended course. FIG. 4B shows an example in which the display control unit 17 displays the staircase 93 in order to notify of a movement course using the staircase 93 as the recommended course. However, the method of displaying the recommended course is not limited to those shown in FIGS. 4A and 4B.

FIGS. 5A and 5B are diagrams showing other display examples of the display device 72. FIG. 5A shows an example in which the display control unit 17 displays the staircase 93 in order to notify of a movement course using the staircase 93 as the recommended course and displays a person walking slowly (while displaying the object person small and displaying an arrow short) in order to notify of slow walking as the recommended speed. FIG. 5B shows an example in which the display control unit 17 displays the staircase 93 in order to notify of a movement course using the staircase 93 as the recommended course and displays a person walking fast (while displaying the object person large and displaying an arrow long) in order to notify of slightly fast walking (fast walking) as the recommended speed. Incidentally, in the case of the slightly fast walking, it is also possible to employ a display method of increasing the object person's leg motion speed in the display. However, the method of displaying the recommended course and the recommended speed is not limited to those shown in FIGS. 5A and 5B.

FIG. 6 is a flowchart showing the operation of the recommended activity presentation device 10 according to the first embodiment. FIG. 6 shows the activity amount management method executed by the recommended activity presentation device 10.

First, the image acquisition unit 11 of the recommended activity presentation device 10 acquires the image data I outputted from the one or more image capturing devices 71 capturing images of the inside of the structure 92 including a plurality of movement courses that can be used by the object person 80 (step S11). Further, the data acquisition unit 12 of the recommended activity presentation device 10 acquires the sensing data S outputted from the physical sensor 81 sensing the physical condition of the object person 80 and the terminal data T for identifying the mobile terminal 82 carried by the object person 80 (step S12). The steps S11 and S12 may also be executed in reverse order or executed in parallel.

Subsequently, the object person identification unit 13 of the recommended activity presentation device 10 identifies the object person 80 based on the image data I and the object person data P indicating the attributes of the object persons 80 and stored in the object person database 61, or based on the terminal data T and the object person data P (step S13).

Subsequently, the activity amount evaluation unit 14 of the recommended activity presentation device 10 acquires or calculates the target activity amount A1 of the object person 80 per the predetermined reference period based on the object person data P, calculates the track record activity amount A2 of the object person 80 from the start time point of the reference period to the present time point based on the sensing data S and the object person data P, and calculates the shortage activity amount A3 as the activity amount by which the object person 80 is short based on the target activity amount A1 and the track record activity amount A2 (step S14).

Subsequently, the course-speed determination unit 15 of the recommended activity presentation device 10 determines the recommended course for the object person 80 among the plurality of movement courses and the recommended speed when moving through the movement course based on the shortage activity amount A3 and the object person data P (step S15). Incidentally, in cases where the object person data P indicates that the object person 80 is a handicapped person or a person having chronic disease, the course-speed determination unit 15 preferentially recommends a movement course including the elevator 94, for example.

Subsequently, the display mode determination unit 16 of the recommended activity presentation device 10 determines the display mode of the notification information to be used when making the display device 72 in the structure 92 display the notification information indicating at least one of the recommended course and the recommended speed (step S16). Subsequently, the display control unit 17 of the recommended activity presentation device 10 makes the display device 72 display the notification information in the recommended display mode (step S17).

As described above, according to the first embodiment, the recommended course and the recommended speed for mitigating the insufficiency of the activity amount of the object person 80 using the structure 92 can be presented to the object person 80.

Further, according to the first embodiment, the display mode of the notification information on the display device 72 can be changed based on the shortage activity amount A3 of the object person 80, and thus it is possible to prompt the object person 80 to do activity with an appropriate activity amount at a place other than an exercise facility.

Furthermore, according to the first embodiment, the recommended course is determined by referring to the object person database, and thus the recommended course and the recommended speed taking the object person 80's physical factor (e.g., presence/absence of a handicap, presence/absence of chronic disease, etc.) into consideration can be presented.

Moreover, according to the first embodiment, the recommended course can be determined while taking an external factor (e.g., movement courses or the like) into consideration.

FIG. 7 is a schematic diagram showing an example of application of a recommended activity presentation device 10a according to a first modification of the first embodiment. In FIG. 7, each component identical or corresponding to a component shown in FIG. 1 is assigned the same reference character as in FIG. 1. While the recommended activity presentation device 10 in the example of FIG. 1 is installed in the structure 92 used by the object person 80, the recommended activity presentation device 10a in the example of FIG. 7 is an external device (e.g., server) capable of communication via the network 83, and a building management device 95 capable of communicating with the recommended activity presentation device 10a is installed in the structure 92.

FIG. 8 is a functional block diagram schematically showing the configuration of the recommended activity presentation device 10a and a recommended activity presentation system la. In FIG. 8, each component identical or corresponding to a component shown in FIG. 3 is assigned the same reference character as in FIG. 3. While the recommended activity presentation device 10 in the example of FIG. 3 is connected to one or more image capturing devices 71 and display devices 72 via the interface 104, the recommended activity presentation device 10a in the example of FIG. 8 executes communication with the building management device 95 via the communication device 105 or via the communication device 105 and the network 83.

As described above, with the recommended activity presentation device 10a shown in FIG. 7 and FIG. 8, the recommended course and the recommended speed for mitigating the insufficiency of the activity amount of the object person 80 using the structure 92 can be presented to the object person 80.

FIG. 9 is a functional block diagram schematically showing the configuration of a recommended activity presentation device 10b and a recommended activity presentation system 1b according to a second modification of the first embodiment. In FIG. 9, each component identical or corresponding to a component shown in FIG. 2 is assigned the same reference character as in FIG. 2. In the example of FIG. 9, the recommended activity presentation device 10b includes a people count measurement unit 18 that measures the number of people included in an image (referred to also as a “people count”), and the course-speed determination unit 15 evaluates a congestion level (an example of the external factor) corresponding to the number of people per unit area and does not recommend a movement course including an area at a high congestion level. Specifically, the course-speed determination unit 15 of the recommended activity presentation device 10b does not recommend a movement course including an area whose congestion level exceeds a predetermined threshold value (e.g., a landing of the staircase, a corridor, or the like).

FIG. 10 is a flowchart showing the operation of the recommended activity presentation device 10b according to the second modification of the first embodiment. In FIG. 10, each step identical or corresponding to a step shown in FIG. 6 is assigned the same number as in FIG. 6. As shown in FIG. 10, the recommended activity presentation device 10b has step S12b of measuring the people count in an image and calculating the congestion level of each area, and determines the notification information based on the shortage activity amount A3 and the congestion level in step S15b of determining the notification information. Except for these features, the process of FIG. 10 is the same as the process of FIG. 6.

As described above, with the recommended activity presentation device 10b shown in FIG. 9 and FIG. 10, the recommended course and the recommended speed for mitigating the insufficiency of the activity amount of the object person 80 using the structure 92, taking the congestion level into consideration, can be presented to the object person 80. Further, since the congestion level is taken into consideration, the object person 80 can reduce useless stops of the walking during the movement.

### Second Embodiment

FIG. 11 is a schematic diagram showing an example of application of a recommended activity presentation device 20 as an activity amount management device according to a second embodiment. In FIG. 11, each component identical or corresponding to a component shown in FIG. 1 is assigned the same reference character as in FIG. 1. The recommended activity presentation device 20 differs from the recommended activity presentation device 10 according to the first embodiment in that the recommended activity presentation device 20 detects a group 280 made up of a plurality of people and determines display content and the display mode for the group 280 when an object person 80 as a person registered in the object person database 61 is included in the group 280.

FIG. 12 is a functional block diagram schematically showing the configuration of the recommended activity presentation device 20 and a recommended activity presentation system 2 as an activity amount management system according to the second embodiment. In FIG. 12, each component identical or corresponding to a component shown in FIG. 2 is assigned the same reference character as in FIG. 2. The recommended activity presentation device 20 is a device capable of executing a recommended activity presentation method as an activity amount management method according to the second embodiment. The recommended activity presentation device 20 is, for example, a computer capable of executing a recommended activity presentation program as an activity amount management program according to the second embodiment. The hardware configuration of the recommended activity presentation device 20 is the same as that shown in FIG. 3.

The recommended activity presentation device 20 differs from the recommended activity presentation device 10 according to the first embodiment in further including a group detection unit 21 that detects the group 280 as a walker group made up of a plurality of people based on the image data I and a group display evaluation unit 22 that sets a recommended speed that can be most easily implemented by all of a plurality of object persons in the group 280 when all of the plurality of object persons move through the same movement course and the display mode.

The group detection unit 21 estimates what people included in the image data I consist a group of people moving and acting together. For the group detection, there have been known a detection method using a relationship among walkers in regard to a walking locus and the direction of attention as clues, a detection method using machine learning, and so forth.

When a plurality of object persons 80 are included in the group 280, the group display evaluation unit 22 determines the notification information (i.e., the combination of the recommended course and the recommended speed) in regard to an object person capable of moving with the smallest activity amount as the notification information for the group 280. That is, when there are a plurality of object persons 80 in the group 280, the course-speed determination unit 15 determines the notification information in regard to an object person whose shortage activity amount A3 is the smallest as the notification information for the group 280. Alternatively, when there are a plurality of object persons 80 in the group 280, the course-speed determination unit 15 may determine the notification information in regard to an object person whose shortage activity amount A3 is the largest as the notification information for the group 280. Further, when there are a plurality of object persons 80 in the group 280, the course-speed determination unit 15 may determine the notification information in regard to an average value of the shortage activity amounts A3 as the notification information for the group 280. Furthermore, when there are a plurality of object persons 80 in the group 280 and the object persons 80 include a wheelchair user, the course-speed determination unit 15 may generate notification information recommending the use of the elevator to a first group including the wheelchair user and recommending the use of the staircase to a second group of the remaining people.

FIG. 13 is a flowchart showing the operation of the recommended activity presentation device 20. In FIG. 13, each step identical or corresponding to a step shown in FIG. 6 is assigned the same number as in FIG. 6.

First, the image acquisition unit 11 acquires the image data I (step S11) and the data acquisition unit 12 acquires the sensing data S and the terminal data T (step S12).

Subsequently, the group detection unit 21 detects a group 280 of walkers (step S21). Subsequently, the object person identification unit 13 identifies one or more object persons 80 in the group 280 (step S22). Subsequently, in regard to each object person 80, the activity amount evaluation unit 14 acquires or calculates the target activity amount A1, calculates the track record activity amount A2, and calculates the shortage activity amount A3 (step S23).

When there is only one object person 80 in the group 280, the course-speed determination unit 15 determines the recommended course and the recommended speed based on the shortage activity amount A3 and the object person data P (step S24). When there are a plurality of object persons 80 in the group 280, the course-speed determination unit 15 determines the notification information (i.e., the combination of the recommended course and the recommended speed) in regard to an object person capable of moving with the smallest activity amount as the notification information for the group 280 (step S24). Incidentally, in cases where the object person data P indicates that the object person 80 is a handicapped person or a person having chronic disease, the course-speed determination unit 15 preferentially recommends a movement course including the elevator 94, for example.

Subsequently, the display mode determination unit 16 determines the display mode to be used when making the display device 72 in the structure 92 display the notification information indicating at least one of the recommended course and the recommended speed (step S16). Subsequently, the display control unit 17 makes the display device 72 display the notification information in the recommended display mode (step S17).

As described above, according to the second embodiment, when a plurality of object persons 80 are included in a group 280 of walkers using the structure 92, the notification information (i.e., the combination of the recommended course and the recommended speed) in regard to an object person capable of moving with the smallest activity amount is determined as the notification information for the group 280. Therefore, it is only necessary to display one piece of notification information for the group of a plurality of people on each display device 72, and each object person 80 can easily recognize the notification information.

Except for the above-described features, the second embodiment is the same as the first embodiment. Further, features in the second embodiment may be applied to the device in the first embodiment.

### Third Embodiment

FIG. 14 is a schematic diagram showing an example of application of a recommended activity presentation device 30 as an activity amount management device according to a third embodiment. In FIG. 14, each component identical or corresponding to a component shown in FIG. 1 is assigned the same reference character as in FIG. 1. The recommended activity presentation device 30 differs from the recommended activity presentation device 10 according to the first embodiment in that the recommended activity presentation device 30 calculates a scheduled activity amount A4 necessary for moving from the entrance of the structure 92 to a destination via a movement course, in regard to each movement course by using destination data D indicating the destination of the object person 80 and determines the recommended course and the recommended speed by using the scheduled activity amount A4 in regard to each movement course.

FIG. 15 is a functional block diagram schematically showing the configuration of the recommended activity presentation device 30 and a recommended activity presentation system 3 as an activity amount management system according to the third embodiment. In FIG. 15, each component identical or corresponding to a component shown in FIG. 2 is assigned the same reference character as in FIG. 2. The recommended activity presentation device 30 is a device capable of executing a recommended activity presentation method as an activity amount management method according to the third embodiment. The recommended activity presentation device 30 is, for example, a computer capable of executing a recommended activity presentation program as an activity amount management program according to the third embodiment. The hardware configuration of the recommended activity presentation device 30 is the same as that shown in FIG. 3.

The recommended activity presentation device 30 differs from the recommended activity presentation device 10 according to the first embodiment in further including a destination database 64 storing the destination data D indicating the destination of the object person 80 and a destination evaluation unit 31 to calculate the scheduled activity amount A4 necessary for moving from the entrance of the structure 92 to the destination via a movement course, in regard to each movement course based on the destination data D regarding the object person 80. The destination evaluation unit 31 acquires the destination of the object person 80 (e.g., the position of the workplace in the structure 92, the floor number of the floor on which the workplace is situated, or the like) from the destination data D regarding the object person 80 acquired by the data acquisition unit 12. The destination evaluation unit 31 calculates the scheduled activity amount A4 necessary for moving from the entrance of the structure 92 to the destination via a movement course, in regard to each movement course. That is, the destination evaluation unit 31 evaluates the scheduled activity amount A4 in regard to each movement course to the destination. The course-speed determination unit 15 determines the recommended course and the recommended speed for reducing the shortage activity amount A3 while taking the evaluation result of the destination evaluation unit 31 into consideration.

FIG. 16 is a flowchart showing the operation of the recommended activity presentation device 30. In FIG. 16, each step identical or corresponding to a step shown in FIG. 6 is assigned the same number as in FIG. 6.

First, the image acquisition unit 11 acquires the image data I (step S11), the data acquisition unit 12 acquires the sensing data S and the terminal data T (step S12), and the destination data D is acquired from the destination database 64 (step S31).

Subsequently, the object person identification unit 13 identifies the object person 80 (step S13). Subsequently, the activity amount evaluation unit 14 acquires or calculates the target activity amount A1, calculates the track record activity amount A2, and calculates the shortage activity amount A3 (step S14). Subsequently, the destination evaluation unit 31 calculates the scheduled activity amount A4 in regard to each movement course to the destination and each walking speed (step S32).

Subsequently, the course-speed determination unit 15 determines the recommended course among the plurality of movement courses and the recommended speed based on the shortage activity amount A3, the object person data P and the scheduled activity amount A4 (step S33). For example, the course-speed determination unit 15 recommends a combination of a recommended course and a recommended speed that leads to a scheduled activity amount A4 closest to the shortage activity amount A3 to the object person 80. Incidentally, in cases where the object person data P indicates that the object person is a handicapped person or a person having chronic disease, the course-speed determination unit 15 preferentially recommends a movement course including the elevator 94, for example.

Subsequently, the display mode determination unit 16 determines the display mode to be used when making the display device 72 in the structure 92 display the notification information indicating at least one of the recommended course and the recommended speed (step S16). Subsequently, the display control unit 17 makes the display device 72 display the notification information in the recommended display mode (step S17).

As described above, according to the third embodiment, the recommended course and the recommended speed for mitigating the insufficiency of the activity amount of the object person 80 using the structure 92 can be acquired while taking each movement course to the destination into consideration, and the notification information with high accuracy can be presented to the object person 80.

Except for the above-described features, the third embodiment is the same as the first embodiment. Further, features in the third embodiment may be applied to the device in the first or second embodiment.

### DESCRIPTION OF REFERENCE CHARACTERS

1, 1a, 1b, 2, 3: recommended activity presentation system, 10, 10a, 10b, 20, 30: recommended activity presentation device, 11: image acquisition unit, 12: data acquisition unit, 13: object person identification unit, 14: activity amount evaluation unit, 15: course-speed determination unit, 16: display mode determination unit, 17: display control unit, 18: people count measurement unit, 21: group detection unit, 22: group display evaluation unit, 31: destination evaluation unit, 61: object person database, 62: movement course database, 63: display model database, 64: destination database, 71: image capturing device, 72: display device, 80: object person, 81: physical sensor, 82: mobile terminal, 83: network, 90: home, 91: bus, 92: structure, 93: staircase, 94: elevator, 95: building management device, 280: group, A1: target activity amount, A2: track record activity amount, A3: shortage activity amount, I: image data, S: sensing data, T: terminal data, P: object person data, M: display model data, D: destination data.

What is claimed is:

1. A recommended activity presentation device for managing an activity amount of an object person, comprising:
processing circuitry
to determine a recommended course as a movement course recommended to the object person among the plurality of movement courses that exist in a structure and can be used by the object person, or a recommended speed as a walking speed recommended when moving through the movement course, or both of the recommended course and the recommended speed based on the shortage activity amount; and
to output the notification information as the recommended course or the recommended speed or both of the recommended course and the recommended speed, to a display device for presenting notification information to the object person.

2. The recommended activity presentation device according to claim 1, wherein the processing circuitry
acquires image data outputted from one or more cameras capturing images of an inside of the structure including the plurality of movement courses;
to acquire sensing data outputted from a physical sensor sensing physical condition of the object person and terminal data for identifying a mobile terminal carried by the object person;
to identify the object person based on the image data and object person data stored in an object person database and indicating attributes of the object person or based on the terminal data and the object person data;
to acquire or calculate a target activity amount of the object person per predetermined reference period based on the object person data, to calculate a track record activity amount of the object person from a start time point of the reference period to a present time point based on the sensing data and the object person data, and to calculate a shortage activity amount as an activity amount by which the object person is short based on the target activity amount and the track record activity amount;
to determine a display mode used when the notification information is displayed by the display device placed in the structure; and
to make the display device display the notification information in the display mode.

3. The recommended activity presentation device according to claim 2, wherein the plurality of movement courses include:
a first course using a staircase in the structure; and
a second course using an elevator in the structure.

4. The recommended activity presentation device according to claim 3, wherein the plurality of movement courses include a third course that includes both of a course using the staircase and a course using the elevator.

5. The recommended activity presentation device according to claim 3, wherein the one or more image cameras include:
a first camera to capture images of a vicinity of an entrance of the structure;
a second camera to capture images of a vicinity of a boarding point of the elevator; and
a third camera to capture images of a vicinity of a landing of the staircase.

6. The recommended activity presentation device according to claim 2, wherein
the physical sensor transmits the sensing data to the mobile terminal, and
the mobile terminal transmits the sensing data to the processing circuitry.

7. The recommended activity presentation device according to claim 2, wherein
the physical sensor is a part of the mobile terminal, and
the mobile terminal transmits the sensing data to the processing circuitry.

8. The recommended activity presentation device according to claim 2, wherein the physical sensor senses one of more out of a number of steps of the object person, body temperature of the object person, and a heart rate of the object person.

9. The recommended activity presentation device according to claim 2, wherein the object person data includes information indicating age, sex and body weight of the object person.

10. The recommended activity presentation device according to claim 3, wherein
the object person data includes information indicating a handicap and disease that the object person has, and
when the object person is judged to be a handicapped person or a person having chronic disease, the processing circuitry determines a movement course using the elevator as the recommended course.

11. The recommended activity presentation device according to claim 2, wherein the object person data includes information indicating travel distance from a start point of the object person to an entrance of the structure and a transportation system used by the object person.

12. The recommended activity presentation device according to claim 2, wherein the processing circuitry
acquires destination data indicating a destination of movement of the object person, and
calculates a scheduled activity amount necessary for moving from an entrance of the structure to the destination via a movement course, in regard to each movement course based on the destination data and determines the recommended movement course and the recommended walking speed by using the scheduled activity amount.

13. The recommended activity presentation device according to claim 2, wherein the processing circuitry
measures a number of people included in an image captured by the image capturing device, and
does not recommend a movement course including an area whose crowd congestion level exceeds a predetermined threshold value.

14. The recommended activity presentation device according to claim 2, wherein the processing circuitry detects a group made up of a plurality of people,
wherein when there are a plurality of object persons in the group, the processing circuitry determines the notification information in regard to an object person whose shortage activity amount is the smallest as the notification information for the group.

15. A recommended activity presentation system for managing an activity amount of an object person, comprising:
one or more cameras to capture images of an inside of a structure including a plurality of movement courses that can be used by the object person;
a physical sensor sensing physical condition of the object person;
a mobile terminal carried by the object person;
a display device provided in the structure;
an object person database storing object person data indicating attributes of the object person; and
processing circuitry
to acquire image data outputted from the one or more cameras,
to acquire sensing data outputted from the physical sensor and terminal data for identifying the mobile terminal,
to identify the object person based on the image data and the object person data or based on the terminal data and the object person data,
to acquire or calculate a target activity amount of the object person per predetermined reference period based on the object person data, to calculate a track record activity amount of the object person from a start time point of the reference period to a present time point based on the sensing data and the object person data, and to calculate a shortage activity amount as an activity amount by which the object person is short based on the target activity amount and the track record activity amount,
to determine a movement course recommended to the object person among the plurality of movement courses and a recommended walking speed when moving through the movement course based on the shortage activity amount and the object person data,
to determine a display mode of notification information to be used when making the display device display the notification information indicating at least one of the recommended movement course and the recommended walking speed, and
to make the display device display the notification information in the display mode.

16. A recommended activity presentation method to be executed by a recommended activity presentation device for managing an activity amount of an object person, comprising:
determining a recommended course as a movement course recommended to the object person among the plurality of movement courses that exist in a structure and can be used by the object person, or a recommended speed as a walking speed recommended when moving through the movement course, or both of the recommended course and the recommended speed based on the shortage activity amount; and
outputting the notification information as the recommended course or the recommended speed or both of the recommended course and the recommended speed, to a display device for presenting notification information to the object person.

17. A non-transitory computer-readable storage medium for storing a recommended activity presentation program that causes a computer managing an activity amount of an object person to execute processes of
determining a recommended course as a movement course recommended to the object person among the plurality of movement courses that exist in a structure and can be used by the object person, or a recommended speed as a walking speed recommended when moving through the movement course, or both of the recommended course and the recommended speed based on the shortage activity amount; and
outputting the notification information as the recommended course or the recommended speed or both of the recommended course and the recommended speed, to a display device for presenting notification information to the object person.
